(19) United States
(12) Patent Application Publication
(10) Pub. No.: US 2008/0140132 A1
(43) Pub. Date: Jun. 12, 2008

(54) Pedicle screw and rod system for minimally invasive spinal fusion surgery

(75) Inventor: Miquelangelo J. Perez-Cruet, Bloomfield, MI (US)

Correspondence Address: MILLER IP GROUP, PLC;MI4 SPINE, LLC, 42690 WOODWARD AVE., SUITE 200, BLOOMFIELD HILLS, MI, 48304, US

(73) Assignee: MI4SPINE, LLC, Bloomfield Village, MI (US)

(21) Appl. No.: 11/635,325

(22) Filed: Dec. 7, 2006

(51) Int. Cl. A61B 17/56; A61B 17/58

(52) U.S. Cl. 606/301; 606/304; 606/264

(57) ABSTRACT

A pedicle screw and rod system for minimally invasive spinal fusion surgery that allows for ease of placement of a fusion rod on the heads of pedicle screws. The system includes extender tubes having a bore extending therethrough. The bore includes a cylindrical portion and a key portion. A slot is formed in the tube along the length of the tube and is open to the bore. The pedicle screws are threaded into the vertebra and the extender tubes are coupled to the heads of the screws so that the slots face each other. The rod is positioned in the slots and is slid down the bores to be coupled to the heads of the pedicle screws. Bolts are slid down the tubes and threaded to the heads of the pedicle screws to secure the rod thereto, and the extender tubes are removed.

### BACKGROUND OF THE INVENTION

1. Field of the Invention

This invention relates generally to a minimally invasive percutaneous spinal instrumentation system that allows pedicle screws and rods to be placed for a spinal fusion procedure without exposing the bony anatomy of the spine by detaching the muscles and ligaments of the spine. More particularly, the invention relates to a pedicle screw and rod system that employs extender tubes having a key portion within the tube that allows a fusion rod to be easily attached to the pedicle screw using minimally invasive spinal fusion surgical techniques.

2. Discussion of the Related Art

The human spine includes a series of vertebrae inter-connected by connective tissue referred to as discs that act as a cushion between the vertebrae. The discs allow for movement of the vertebrae so that the back can bend and rotate.

Spinal fusion is a surgical procedure that fuses two or more vertebrae together using bone grafts and/or other devices. Spinal fusion is a commonly performed procedure for the treatment of chronic neck and back pain refractory to non-operative treatments. Spinal fusion is used to stabilize or eliminate motion of vertebrae segments that may be unstable, i.e., move in an abnormal way, that may lead to pain and discomfort. Spinal fusion is typically performed to treat injuries to the vertebrae, degeneration of the spinal discs, abnormal spinal curvature and a weak or unstable spine.

Spinal instrumentation is a technique known to improve fusion rates resulting in better patient outcomes. The goal of the instrumentation is to prevent the spine from moving at the fused segment, thus much like applying a cast to a broken arm, the instrumentation allows the bone to heal between the fused vertebrae. However, in order to perform traditional spinal instrumentation, the muscle and ligaments need to be stripped off of the spine to expose the bone of the spine. This in itself can lead to additional pain, longer recoveries and failed fusion surgery.

In an attempt to preserve normal anatomical structures during spine fusion surgery, minimally invasive surgical procedures have been devised that do not require disruption of the muscles and ligaments. The spinal instrumentation is placed through small incisions or percutaneously, thus resulting in less blood loss, less post-operative pain, faster recoveries, and improved outcomes. One such procedure involves the placement of Kirschner wires (K-wires) through the skin into the vertebral pedicles of the spine using intra-operative fluoroscopic images to guide the proper placement of the K-wires. The K-wires that are passed through the center of the pedicle of the spine and into the vertebral body act as guide pins to placing the cannulated pedicle screws, which easily slide over the K-wires. Once the pedicle screws are in place, a rod is used to connect the pedicle screws between adjacent segments, thus holding the segments together firmly and allowing bony fusion to occur.

Spinal fusion requires a graft material, usually bone material, to fuse the vertebrae together. The bone graft material can be placed over the spine to fuse adjacent vertebrae together. Alternatively, a cage is positioned between the vertebrae being fused, and is filled with the graft material. This procedure is referred to as interbody fusion since it is between adjacent vertebrae. The cage includes holes that allow the vertebrae and the graft material to grow together to provide the fusion. The cage supports the weight of adjacent vertebrae while the fusion is occurring through the cage. Alternatively, the bone graft material can be placed directly over or lateral to the spine, referred to as postero-lateral fusion. Typically the bone graft material is autogenous bone material taken from the patient, or allograft bone material harvested from cadavers. Synthetic bone materials can also be used as the graft material. Generally, the patient's own bone material offers the best fusion material and is the current “gold standard”.

One of the most common forms of spinal instrumentation is the pedicle screw and rod construct. The rods, which span adjacent vertebrae, are mounted to the vertebra using pedicle screws that are threaded through the pedicles of each vertebra and into the vertebral body. Accurate placement of the pedicle screws relative to the vertebral pedicle is very important to prevent injury to nerves or spinal cord.

One known system for placement of the rod during spinal fusion surgery is referred to as the Pathfinder system that employs extender tubes. The surgeon will connect the extender tubes to the heads of the pedicle screws so that the extender tubes extend out of the patient, where the extender tubes include slots that face each other. An opener device is then slid down the extender tubes to open a pathway between the tubes for the rod through the patient's tissue. A rod placement tool is attached to the rod, and the rod is slid down the slots and is connected to the heads of the pedicle screws. A bolt is then slid down the tube and is threaded to the head of the pedicle screw so that the rod is fixedly attached thereto. The extender tubes are then detached from the head of the pedicle screws, and removed from the patient.

Although the Pathfinder system has been successful in providing rod placement for minimally invasive spinal fusion surgery, improvements can be made to the system, such as by providing an easier technique for inserting the rod through the extender tubes.

### SUMMARY OF THE INVENTION

In accordance with the teachings of the present invention, a pedicle screw and rod system for minimally invasive spinal fusion surgery is disclosed that allows for ease of placement of a fusion rod on the heads of pedicle screws. The system includes extender tubes having a bore extending therethrough. The bore includes a cylindrical portion and a key portion. A slot is formed in the tube along the length of the tube and is open to the bore. The pedicle screws are threaded into the vertebra and the extender tubes are coupled to the heads of the screws so that the slots face each other. The rod is positioned in the slots and is slid down the bores to be coupled to the heads of the pedicle screws. Bolts are slid down the tubes and threaded to the heads of the pedicle screws to secure the rod thereto, and the extender tubes are removed.

Additional features of the present invention will become apparent from the following description and appended claims, taken in conjunction with the accompanying drawings.

### BRIEF DESCRIPTION OF THE DRAWINGS

FIG. 1 is a perspective view of an extender tube employed in a pedicle screw and rod system, according to an embodiment of the present invention;

FIG. 2 is a top view of the extender tube shown in FIG. 1;

FIG. 3 is a bottom view of the extender tube shown in FIG. 1;

FIG. 4 is a broken-away, top perspective view of a pedicle screw that is part of the pedicle screw and rod system of the invention;

FIG. 5 is a side view of the pedicle screw and rod system of the invention including two of the extender tubes shown in FIG. 1 and two pedicle screws attached thereto;

FIG. 6 is a cross-sectional view of the pedicle screw and rod system shown in FIG. 5; and

FIG. 7 is a side view of the pedicle screw and rod system shown in FIG. 5 attached to adjacent vertebra of a patient.

### DETAILED DESCRIPTION OF THE EMBODIMENTS

The following discussion of the embodiments of the invention directed to a pedicle screw and rod system for minimally invasive spinal instrumentation for fusion surgery is merely exemplary in nature, and is in no way intended to limit the invention or its applications or uses.

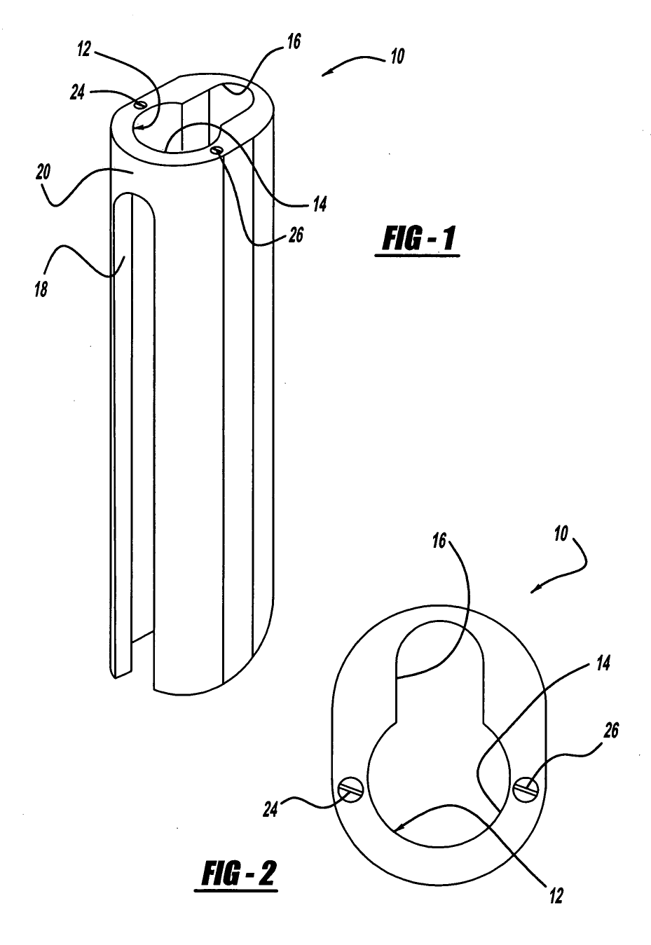

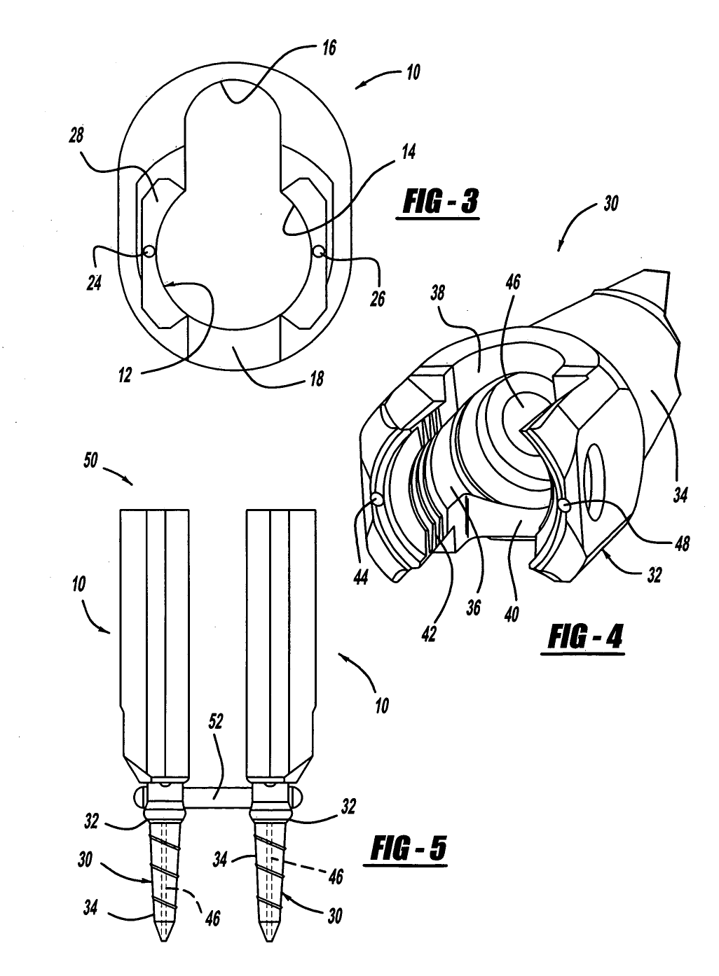

FIG. 1 is a perspective view, FIG. 2 is a top view and FIG. 3 is a bottom view of an extender tube 10 that is part of a pedicle screw and rod system (FIG. 5), according to an embodiment of the present invention. The extender tube 10 extends above the level of the skin incision during the surgical procedure. The extender tube 10 includes an internal channel 12 having a cylindrical portion 14 and a key portion 16. The key portion 16 has a generally rounded configuration to accommodate the end of a cylindrical fusion rod that is slid down the tube 10, as will become apparent from the discussion below. A slot 18 is formed in a sidewall 20 of the extender tube 10, and extends from a bottom of the extender tube 10 to almost to a top of the extender tube 10. The extender tube 10 includes threaded securing rods 24 and 26 having screw heads provided on opposite sides of the cylindrical portion 14, as shown, to attach and detach the extender tube 10 to a pedicle screw, where the rods 24 and 26 extend the length of the tube 10, as will be discussed in further detail below. The cylindrical portion 14 of the channel 12 has a specific cut-out portion 28 at the bottom to accommodate the head of a pedicle screw, as will also further be discussed below.

FIG. 4 is a top perspective view of a pedicle screw 30 that is part of the pedicle screw and rod system of the invention. The pedicle screw 30 includes a pedicle screw head 32 and a pedicle screw body 34 having screw threads. The pedicle head screw head 32 includes a generally cylindrical bore 36 having opposing slots 38 and 40 extending through the screw head 32 on opposite sides of the bore 36, as shown. Further, the pedicle screw head 32 includes a threaded portion 42 between the slots 38 and 40. Threaded holes 44 and 46 accept threaded ends of the rods 24 and 26, respectively, to secure the extender tube 10 to the pedicle screw 30. A bore 46 extends through the screw body 34 to make the screw 30 cannulated allowing the pedicle screw 30 to be placed into the pedicle of the spine in a percutaneous manner over a K-wire that was previously placed through the pedicle.

FIG. 5 is a side view and FIG. 6 is a cross-sectional view of a pedicle screw and rod system 50, according to an embodiment of the invention. The system 50 includes two of the extender tubes 10 and two of the pedicle screws 30, where the slots 18 in the extender tubes 10 face each other. A cylindrical fusion rod 52 is positioned within the channels 12 using a suitable tool 9 not shown) that advances the rod 52 down the extender tubes 10 from the top of the skin to position onto the heads 32 of the pedicle screws 30 that have been placed in the spine. Rounded ends of the rod 52 are positioned within the key portions 16 so that the rod 52 can be slid down the extender tubes 10 evenly without having to send one end of the rod 52 down an extender tube 10 first in a back and forth motion, as was required using the extender tubes in the Pathfinder system. The rod 52 is slid down the channels 12 and is positioned within the slots 38 and 40 in the pedicle screw heads 32, as shown, so that ends of the rod 52 extend across the slots 38 and 40 and out of the head 32. Thus, the key portion 16 allows the desired length of the rod 52 to be accommodated within the extender tubes 10.

Bolts 54 are then slid down the channels 12 using a suitable tool (not shown) and are threaded to the pedicle screw head 32 using the threaded portion 42. Thus, the fusion rod 52 is securely attached to the pedicle screws 30 to achieve a solid construct for spinal fusion. The threaded rods 24 and 26 are then unthreaded from the holes 44 and 48 to release the extender tubes 10 from the pedicle screw heads 32 so that they can be removed from the patient leaving the pedicle screw rod system 50 in place.

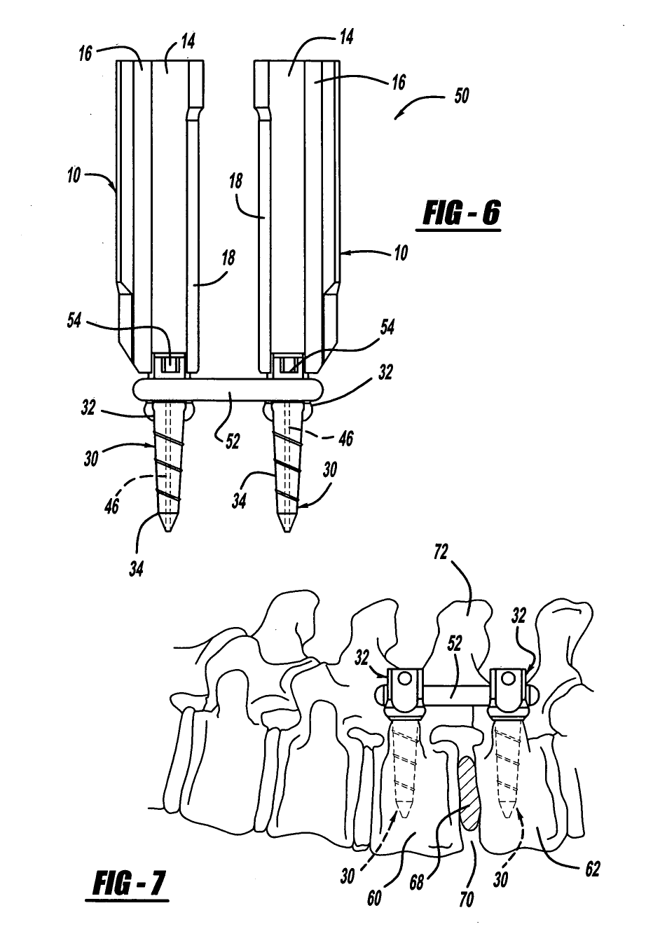

FIG. 7 is a side view of the pedicle screw and rod system 50 where the pedicle screws 30 have been threaded through the pedicles of vertebra 60 and 62. The extender tubes 10 have been removed, as discussed above, where the pedicle screws 30 and the rod 52 will remain in the patient's body to provide the a stable construct to enhance fusion along with a bone graft device 68 positioned within the disc space 70, as is well understood to those skilled in the art. A second pedicle screw and rod system (not shown) would be provided on the other side of the spinous process 72 of the vertebra 60 and 62.

The foregoing discussion discloses and describes merely exemplary embodiments of the present invention. One skilled in the art will readily recognize from such discussion and from the accompanying drawings and claims that various changes, modifications and variations can be made therein without departing from the spirit and scope of the invention as defined in the following claims.

1. A pedicle screw and rod system comprising:
at least one extender tube including a channel extending therethrough, said channel including a cylindrical portion and a key portion, said cylindrical portion and said key portion being parallel and open to each other, said extender tube further including a slot extending a portion of the length of the extender tube and being open to the channel opposite to the key portion; and
at least one pedicle screw including a pedicle screw body and a pedicle screw head, wherein a portion at the bottom of the channel in the extender tube is configured to conform to the shape of the pedicle screw head so that the pedicle screw head can be positioned within the channel.

2. The system according to claim 1 wherein the slot extends almost the entire length of the extender tube.

3. The system according to claim 1 wherein the key portion has a rounded configuration.

4. The system according to claim 1 wherein the at least one extender tube is two extender tubes and the at least one pedicle screw is two pedicle screws.

5. The system according to claim 4 further comprising a fusion rod being operable to be coupled to the heads of the pedicle screws and having a shape and length to extend into the key portion of the two extender tubes through the slots.

6. The system according to claim 5 wherein the fusion rod extends through cut-out portions in the heads of the pedicle screws.

7. The system according to claim 5 wherein the fusion rod is operable to be coupled to the heads of the pedicle screws by bolts.

8. The system according to claim 1 wherein the at least one extender tube is coupled to the at least one pedicle screw by at least one securing rod that extends the length of the at least one extender tube.

9. A pedicle screw and rod system for fusing vertebra together, said system comprising:
a pair of extender tubes each including a channel extending therethrough, said channel including a cylindrical portion and a key portion, said cylindrical portion and said key portion being parallel and open to each other, each extender tube further including a slot extending a portion of the length of the extender tube and being open to the channel opposite to the key portion;
a pair of pedicle screws each including a pedicle screw body and a pedicle screw head, each pedicle screw head including an inner chamber and a cut-out portion extending across the chamber, said pedicle screw head further including a threaded portion within the inner chamber, wherein a portion of the channel in each extender tube is configured to conform to the shape of the pedicle screw head so that the pedicle screw head can be positioned within the chamber so as to attach the extender tube to the pedicle screw; and
a fusion rod operable to be coupled to the heads of the pedicle screws and having a shape and length to fit into the key portion of the extender tubes through the slots.

10. The system according to claim 9 wherein the slot extends almost the entire length of the extender tube.

11. The system according to claim 9 wherein the key portion has a rounded configuration.

12. The system according to claim 9 wherein the fusion rod is operable to be coupled to the heads of the pedicle screws by bolts threaded into the threaded portion.

13. The system according to claim 9 wherein the extender tube is removably coupled to the pedicle screw by at least one securing rod that extends the length of the extender tube.

14. An extender tube comprising an elongated body portion, said elongated body portion including a bore extending therethrough, said bore including a cylindrical portion and a key portion, said cylindrical portion and said key portion being parallel and open to each other, said body portion further including a slot extending a portion of the length of the body portion and being open to the bore opposite to the key portion.

15. The tube according to claim 14 wherein the slot extends almost the entire length of the extender tube.

16. The tube according to claim 14 wherein the key portion has a rounded configuration.

17. The tube according to claim 14 wherein the extender tube is part of a pedicle screw and rod system used to fuse two vertebra together.

18. The system according to claim 8 wherein the at least one securing rod includes a threaded end portion that is operable to be threaded into a threaded opening in the pedicle screw head and wherein the at least one securing rod is operable to be threaded and unthreaded from an opposite end of the securing rod from the pedicle screw head.

19. The system according to claim 13 wherein the at least one securing rod includes a threaded end portion that is operable to be threaded into a threaded opening in the pedicle screw head and wherein the at least one securing rod is operable to be threaded and unthreaded from an opposite end of the securing rod from the pedicle screw head.
